(19) United States
(12) Patent Application Publication
(10) Pub. No.: US 2026/0191782 A1
(43) Pub. Date: Jul. 9, 2026

(54) URIDINE TRIACETATE AMORPHOUS FORMULATION AND USES THEREOF

(71) Applicant: PHARMA CINQ, LLC, Rockville, MD (US)

(72) Inventors: Michael Kevin BAMAT, Potomac, MD (US); Yi GAO, Vernon Hills, IL (US); Jeffrey A. MILLER, Lincoln University, PA (US); Yihong QIU, Vernon Hills, IL (US); Reid W. VON BORSTEL, Potomac, MD (US)

(21) Appl. No.: 18/998,209

(22) PCT Filed: Jul. 26, 2023
(86) PCT No.: PCT/US2023/000027

§ 371 Date: Jan. 24, 2025

Related U.S. Application Data
(60) Provisional application No. 63/392,304, filed on Jul. 26, 2022.

(51) Int. Cl. A61K 9/14; A61K 31/7072; A61P 3/00

(52) U.S. Cl. CPC A61K 9/146; A61K 31/7072; A61P 3/00

(57) ABSTRACT

There is disclosed a dispersion of amorphous uridine triacetate in Hypromellose Acetate Succinate-MG and optionally also Copovidone. The amorphous dispersion compositions allow high loading of uridine. They also have good stability and oral bioavailability. They can be used to deliver exogenous uridine to a mammalian subject in need thereof, for example a subject who has an energy failure disorder or certain other conditions.

### BACKGROUND OF THE INVENTION

The pyrimidine nucleoside uridine has several potential clinical applications, including treatment of fluoropyrimidine toxicity and mitochondrial energy failure disorders. A barrier to therapeutic use of uridine itself is poor oral bioavailability, which has been measured at approximately 7% to 10% in both humans and mice. Uridine triacetate, an ester prodrug of uridine, in its crystalline form, improves oral bioavailability to approximately 50%. However, because relatively large doses of uridine triacetate are required for its therapeutic use in some disorders, up to 5 to 10 grams per dose, further improvement in bioavailability is important, both for reducing the amount of drug needed (and corresponding costs), and especially for therapeutic indications that benefit from a high peak concentration (Cmax) of plasma uridine, such as mitochondrial diseases and other energy failure disorders including but not limited to Huntington's Disease, Down Syndrome Dementia, age-related dementia and neuromuscular degeneration (sarcopenia), and vulnerability to secondary injury after acute brain injury such as traumatic brain injury, concussions, ischemic or hemorrhagic strokes, and asphyxia.

One important factor regulating the rate and extent of delivery of plasma uridine after oral administration of uridine triacetate is the rate of dissolution of crystals of this compound. Uridine triacetate has limited solubility in water, approximately 10 milligrams per milliliter.

Because relatively large doses of uridine triacetate are required, a high ratio of uridine triacetate to formulation excipients is important; however, a high ratio of uridine triacetate to excipients may result in poor stability, including crystal formation over time, creating both functional and regulatory problems. Furthermore, excipients must be safe in quantities compatible with the requisite amounts of uridine triacetate.

### SUMMARY OF THE INVENTION

This invention provides a composition comprising uridine triacetate formulated as an amorphous (noncrystalline) dispersion in one or more excipients, wherein the amount of the amorphous uridine triacetate is from about fifty to about sixty percent by weight of the composition; one of the one or more excipients is Hypromellose Acetate Succinate-MG; and the amount of the Hypromellose Acetate Succinate-MG is from about thirty-seven to about forty percent by weight of the composition.

This invention provides a method of delivering exogenous uridine to a mammalian subject in need thereof, comprising administering an effective amount of the composition to the subject. The method of this invention is useful in treating a mammalian subject who has a uridine deficiency condition or an energy failure disorder; or who is receiving fluoropyrimidine chemotherapy and the exogenous uridine modulates toxicity or efficacy, or both toxicity and efficacy of the fluoropyrimidine chemotherapy.

### DETAILED DESCRIPTION OF THE INVENTION

In an embodiment of the composition according to this invention, one of the one or more excipients is Copovidone. When the composition comprises Copovidone it is convenient for the amount of Copovidone to be about twelve percent by weight of the composition, for example about 12.5 percent by weight.

In a preferred embodiment of the composition according to this invention, the amount of amorphous uridine triacetate is about sixty percent by weight of the composition and the amount of the Hypromellose Acetate Succinate-MG is about forty percent by weight of the composition. In another preferred embodiment the amount of amorphous uridine triacetate is about fifty percent by weight of the composition, and the one or more excipients comprise Hypromellose Acetate Succinate-MG in an amount of about 37.5 percent by weight of the composition and Copovidone in an amount of about 12.5 percent by weight of the composition.

The amorphous dispersion compositions according to this invention allow high loading of uridine triacetate, adequate stability during storage, and improved oral bioavailability in comparison to equimolar doses of crystalline uridine triacetate particles. Moreover the compositions in accordance with this invention have improved taste and texture compared to the coated granules of crystalline uridine triacetate currently being commercialized. The compositions in accordance with this invention can optionally be mixed with soft foods such as applesauce, pudding or yogurt up to about thirty minutes before being ingested.

Generally, amorphous formulations are used when the active pharmaceutical ingredient (API) is very sparingly soluble and thus small amounts of API are made more soluble and thus bioavailable. It is not generally used with APIs, such as uridine triacetate, which are moderately soluble. An amorphous formulation of uridine triacetate allows practical oral delivery of a large amount of API. In the expressions “amorphous formulation” and “amorphous dispersion” of uridine triacetate, the term “amorphous” refers to the fact of the uridine triacetate being non-crystalline.

Amorphous dispersions are produced by one of two basic methods, spray drying or hot melt extrusion. In spray drying, the drug and excipients (generally including a polymer) are dissolved in a volatile solvent. The solution is sprayed as a fine mist and solvent is evaporated by heat or vacuum, leaving fine particles that are collected. In hot melt extrusion, the drug and excipients are melted together, mixed, extruded and cooled, yielding a solid material that can be milled to form particles of suitable size. In accordance with this invention the particles can be milled to any conventional size. For example it is convenient for the particles to have a D50 of about 200 microns.

Amorphous dispersion particles are optionally further formulated into aggregates, coated with taste masking or modified release excipients. Particles can also be incorporated into suspensions, capsules or tablets, including miniature tablets that are small enough to pass through gastrostomy or nasogastric tubes, or to be administered via an oral dosing syringe.

Uridine triacetate readily crystallizes under aqueous conditions. Therefore it was unexpected that hot melt extrusion compositions, such as formulation 1 (60% API/40% HPMCAS-MG) and formulation 2 (50% API/37.5% HPMCAS-MG/12.5% Copovidione), could be made successfully, display stability and overcome the challenge of targeting a very high drug load (≥50% uridine triacetate) of an API prone to reverting to a crystallized form, especially in the presence of moisture.

In the field of hot melt extrusion it is common to add a surfactant or plasticizer. Nevertheless it was found that the hot melt extrusion of the formulations according to this invention did not require addition of a surfactant or plasticizer (Example 1 and Example 2). Without wishing to be bound by theory, it appears that uridine triacetate itself may be acting as a plasticizer.

Compositions of this disclosure are beneficial in disorders or diseases for which delivery of exogenous uridine is beneficial, which can include uridine deficiency states (e.g. hereditary orotic aciduria or biallelic CAD deficiency), modulation of toxicity and/or efficacy of fluoropyrimdine chemotherapy, and a variety of energy failure conditions, particularly those involving either genetic or acquired mitochondrial dysfunction. Compositions of this disclosure are specifically intended to optimize treatment of energy failure disorders, which comprise:
1. Primary mitochondrial diseases (PMD), with pathogenic mutations in either mitochondrial DNA or nuclear DNA that impair mitochondrial energy production.
2. Chronic neurodegenerative diseases featuring pathogenic deficits in mitochondrial function, including but not limited to Huntington's Disease, Alzheimer's Dementia, Parkinson's Disease, Amyotrophic Lateral Sclerosis (ALS), Down Syndrome Dementia.
3. Neuromuscular dysfunction or wasting featuring pathogenic mitochondrial dysfunction including sarcopenia (age-related or earlier onset exacerbated by chronic kidney disease, COPD or heart failure), cachexia, muscle disuse atrophy, and circulatory insufficiency (e.g. peripheral artery disease or intermittent claudication)
4. Acute brain injury, including but not limited to traumatic brain injury, stroke (both ischemic and hemorrhagic), birth asphyxia, cardiac arrest, drowning and carbon monoxide poisoning.
5. Acute or chronic myocardial ischemia, left ventricular heart failure, right ventricular heart failure (e,g, due to pulmonary arterial hypertension).

The common feature among these energy failure disorders along successful treatment with a composition of this disclosure is the presence of genetic or acquired mitochondrial dysfunction resulting in impairment of mitochondrial reserve (or spare) energy capacity, the ability to rapidly increase cellular energy production in response to demand, a crucial property, since ATP is not stored but must be continually generated by mitochondrial oxidative phosphorylation and glycolysis. A gap between energy production and utilization is a primary determinant of cellular dysfunction and death across a large variety of diseases and conditions exemplified by those listed above.

For treatment of energy failure disorders, a primary pharmacokinetic and pharmacodynamic goal is to deliver sufficient uridine into cells to augment intracellular, and specifically intramitochondrial, uridine nucleotides, especially within the mitochondrial intermembrane space (IMS). Without being bound to a specific mechanism of action, one contribution of exogenous uridine to improve mitochondrial bioenergetic capacity is activation of the mitochondrial ATP-sensitive potassium channel in the mitochondrial inner membrane by elevation of concentrations of uridine diphosphate (UDP) in the mitochondrial intermembrane space (IMS). UTP (uridine triphosphate) and UDP are in equilibrium with the ratio of ATP to ADP via the enzyme nucleoside diphosphate kinase, which spans the inner and outer mitochondrial membranes in the IMS. The ratio of ATP/ADP and therefore UTP/UDP acts as an index of cellular bioenergetic state, with elevation of ADP and UDP in the IMS occurring when either mitochondrial ATP synthesis is impaired or cytosolic energy utilization is increased to where it exceeds the rate of replenishment by oxidative phosphorylation or glycolysis. Augmentation of total IMS uridine nucleotides with exogenous uridine delivered by uridine triacetate in a composition of this disclosure enables more rapid and extensive elevation of UDP in the IMS when cellular bioenergetic capacity is diminished. Activation of the mitochondrial ATP-sensitive potassium channel by UDP prevents disruption of optimum mitochondrial architecture during impending energy failure, specifically attenuating osmotic expansion of the width of the IMS and concurrent shrinkage of the mitochondrial matrix, the interior compartment of mitochondria. IMS width is a decisive determinant of the rate of fuel oxidation and of efficiency of transfer of bioavailable energy from ATP within mitochondria to creatine phosphate in the cytosol; the latter enables more rapid equilibration of phosphorylation (energy) potential throughout a cell. Efficient function of mitochondrial Creatine Kinase (CK), which enables retention of ATP+ADP in mitochondria while exporting Creatine Phosphate (CrP) generated from creatine to cytosolic sites (where ATP is locally regenerated from CrP), depends on maintenance of the width of the IMS at about 90 Angstroms so that the enzyme physically bridges the inner and outer mitochondrial membranes. By facilitating maintenance of IMS width, compositions of this disclosure mitigate deterioration of reserve energy capacity in energy failure disorders, reducing cellular dysfunction and improving health and survival. The energy state-dependence of production of UDP in the IMS enables chronic treatment of energy failure disorders without the adverse consequences of other classes of drugs, which can open the ATP-sensitive channel but do so even when cellular energy is replete, resulting in inappropriate swelling of the mitochondrial matrix and narrowing of the IMS width below optimum distances, which can impair oxidative phosphorylation. Compositions of this disclosure therefore have a unique advantage over other modulators of mitochondrial ATP-sensitive potassium channel activity, especially for chronic treatment of energy failure disorders.

Optimum single doses of compositions of this disclosure for treatment of energy failure disorders in human patients comprise 30 to 120 mg/kg uridine triacetate (plus polymeric and other excipients), more specifically 60 to 100 mg/kg uridine triacetate (for example as the active agent in 100 to 167 mg/kg of the composition with 60% uridine triacetate in Example 1 below, or 120 to 200 mg/kg of the composition with 50% uridine triacetate loading in Example 2 below). 1 to 4 doses per day are administered orally, generally as two doses per day separated by approximately 8 to 12 hours.

For treatment of disorders involving the central nervous system, the pharmacokinetic goal is to saturate uridine transport across the blood-brain barrier (largely via endothelial pyrimidine transporters of the ENT family), achieving peak plasma uridine concentrations exceeding 100 micromolar. Optimum treatment of peripheral organs may be achieved at lower concentrations of plasma uridine than is required for treatment of the brain, due to the absence of a restrictive epithelial barrier comparable to the blood-brain barrier in most other tissues.

### ABBREVIATIONS AND DEFINITIONS

HPMCAS means Hypromellose Acetate Succinate (a/k/a Hydroxypropyl Methyl Cellulose Acetate Succinate).
KF refers to the Karl Fischer method for the determination of moisture content.
RH means Relative Humidity.
CAD is an enzyme complex catalyzing the first committed steps in pyrimidine nucleotide biosynthesis; Carbamoyl-phosphate synthetase 2, Aspartate transcarbamoylase, and Dihydroorotase.
Uridine triacetate is also known as 2′,3′,5′-Tri-O-acetyluridine or triacetyluridine.

The invention will be better understood by reference to the following examples, which illustrate but do not limit the invention described herein.

### EXAMPLES

### Example 1: Amorphous Solid Dispersion Consisting of 60% Uridine Triacetate and 40% Hypromellose Acetate Succinate-MG

An amorphous solid dispersion (ASD) consisting of 60% Uridine Triacetate (w/w) active pharmaceutical ingredient (API) and 40% Hypromellose Acetate Succinate-MG (HPMCAS-MG) polymer was prepared by mixing Uridine Triacetate with Hydroxypropyl Methyl Cellulose Acetate Succinate-MG in ratios as described in Table 1, followed by hot melt extrusion with a twin screw extruder. The cooled extrusion was milled to a D50 of approximately 200 microns.

TABLE 1
Materials for 40% Uridine Triacetate and
60% Polymer Amorphous Solid Dispersion

| | | % of | |
|---|---|---|---|
| Chemical Name | Trade Name | Formulation | Source |
| Uridine Triacetate | NA | 60% | Wellstat |
| Hypromellose | AquaSolve ™ | 40% | Ashland |
| Acetate Succinate-MG | HPMCAS-MG | | |

### Example 2: Amorphous Solid Dispersion Consisting of 50% Uridine Triacetate and 37.5% Hypromellose Acetate Succinate-MG and 12.5% Copovidone

An amorphous solid dispersion (ASD) consisting of 50% Uridine Triacetate (w/w) active pharmaceutical ingredient (API), 37.5% Hypromellose Acetate Succinate-MG (HPMCAS-MG) and 12.5% Copovidone was prepared by mixing Uridine Triacetate with Hypromellose Acetate Succinate-MG and Copovidone polymers in the ratios as described in Table 2, followed by hot melt extrusion with a twin screw extruder. The cooled extrusion was milled to a D50 of approximately 200 microns.

TABLE 2
Materials for 60% Uridine Triacetate and
40% Polymer Amorphous Solid Dispersion

| | | % of | |
|---|---|---|---|
| Chemical Name | Trade Name | Formulation | Source |
| Uridine Triacetate | NA | 50% | Wellstat |
| Hypromellose | AquaSolve ™ | 37.5% | Ashland |
| Acetate Succinate-MG | HPMCAS-MG | | |
| Poly(1- | Copovidone 35 M3 | 12.5% | BASF |
| vinylpyrrolidone-co- | H. EUR./NF Type K28 | | |
| Vinyl Acetate) | | | |

### Example 3: Stability of ASD Formulations 1 and 2 Under Long Term (25° C./60% RH) and Accelerated (40° C./75% RH) Conditions

Formulation 1 (60% API) was stable for 12 weeks under long term (Table 3: 25° C./60% RH) and accelerated conditions (Table 4:40° C./75% RH) with no increase in impurities or crystallinity as detected by HPLC and light microscopy, respectively. Formulation 2 (50% API) was stable for 12 weeks under long term (Table 5: 25° C./60% RH) and accelerated conditions (Table 6:40° C./75% RH) with no increase in impurities or crystallinity as detected by HPLC and light microscopy, respectively.

TABLE 3
Formulation 1 (60% API): 25° C./60% RH Stability

| Test | Initial | 4 Weeks | 6 Weeks | 12 Weeks |
|---|---|---|---|---|
| Appearance and | Off-white | Off-white | Off-white | Off-white |
| Color | granules | granules | granules | granules |
| Assay | 100.9% | 98.3% | 99.9% | 100.1% |
| Impurities | | | | |
| Uridine | ND | ND | ND | ND |
| 2′5′ diacetyluridine | <0.03% | 0.04% | <0.03% | 0.03% |
| | (0.02%) | | | |
| 2′-acetyluridine + 3′- | ND | ND | ND | ND |
| acetyluridine | | | | |
| 5′-acetyluridine | ND | ND | ND | ND |
| 2′,3′-diacetyluridine | 0.04% | 0.08% | 0.03% | 0.06% |
| 2′,5′- | 0.05% | 0.10% | <0.03% | 0.07% |
| diacetyluridine + | | | | |
| 3′,5′-diacetyluridine | | | | |
| Individual Unknown | ND | ND | ND | ND |
| Impurity | | | | |
| Total Impurities | 0.09% | 0.22% | 0.03% | 0.16% |
| Crystallinity | No | No | No | No |
| | Crystallinity | Crystallinity | Crystallinity | Crystallinity |
| | Detected | Detected | Detected | Detected |
| Moisture by KF | 0.73% | 0.61% | 0.56% | |

TABLE 4
Formulation 1 (60% API): 40° C./75% RH Stability

| Test | Initial | 4 Weeks | 6 Weeks | 12 Weeks |
|---|---|---|---|---|
| Appearance and | Off-white | Off-white | Off-white | Off-white |
| Color | granules | granules | granules | granules |
| Assay | 100.9% | 98.4% | 100.3% | 100.0% |
| Impurities | | | | |
| Uridine | ND | ND | ND | ND |
| 2′,5′-diacetyluridine | <0.03% | 0.04% | 0.03% | 0.06% |
| | (0.02%) | | | |
| 2′-acetyluridine + 3′- | ND | ND | ND | ND |
| acetyluridine | | | | |
| 5′-acetyluridine | ND | ND | ND | ND |
| 2′,3′-diacetyluridine | 0.04% | 0.09% | 0.05% | 0.14% |
| 2′,5′- | 0.05% | 0.10% | 0.07% | 0.16% |
| diacetyluridine + | | | | |
| 3′,5′-diacetyluridine | | | | |
| Individual Unknown | ND | ND | ND | ND |
| Impurity | | | | |
| Total Impurities | 0.09% | 0.232% | 0.15% | 0.36% |
| Crystallinity | No | No | No | No |
| | Crystallinity | Crystallinity | Crystallinity | Crystallinity |
| | Detected | Detected | Detected | Detected |
| Moisture by KF | 0.73% | 0.60% | 0.56% | |

TABLE 5
Formulation 2 (50% API): 25° C./60% RH Stability

| Test | Initial | 4 Weeks | 6 Weeks | 12 Weeks |
|---|---|---|---|---|
| Appearance and | Off-white | Off-white | Off-white | Off-white |
| Color | granules | granules | granules | granules |
| Assay | 100.5% | 98.9% | 100.9% | 100.2% |
| Impurities | | | | |
| Uridine | ND | ND | ND | ND |
| 2′,5′-diacetyluridine | 0.03% | 0.04% | <0.03% | 0.04% |
| 2′-acetyluridine + 3′- | ND | ND | ND | ND |
| acetyluridine | | | | |
| 5′-acetyluridine | ND | ND | ND | ND |
| 2′,3′-diacetyluridine | 0.05% | 0.07% | 0.03% | 0.07% |
| 2′,5′- | 0.07% | 0.09% | <0.03% | 0.09% |
| diacetyluridine + | | | | |
| 3′,5′-diacetyluridine | | | | |
| Individual Unknown | ND | ND | ND | ND |
| Impurity | | | | |
| Total Impurities | 0.15% | 0.20% | 0.03% | 0.20% |
| Crystallinity | No | No | No | No |
| | Crystallinity | Crystallinity | Crystallinity | Crystallinity |
| | Detected | Detected | Detected | Detected |
| Moisture by KF | 0.88% | 0.78% | 0.76% | |

TABLE 6
Formulation 2 (50% API): 40° C./75% RH Stability

| Test | Initial | 4 Weeks | 6 Weeks | 12 Weeks |
|---|---|---|---|---|
| Appearance and | Off-white | Off-white | Off-white | Off-white |
| Color | granules | granules | granules | granules |
| Assay | 100.5% | 98.8% | 100.5% | 100.8% |
| Impurities | | | | |
| Uridine | ND | ND | ND | ND |
| 2′,5′-diacetyluridine | 0.03% | 0.05% | 0.03% | 0.08% |
| 2′-acetyluridine + 3′- | ND | ND | ND | ND |
| acetyluridine | | | | |
| 5′-acetyluridine | ND | ND | ND | ND |
| 2′,3′ -diacetyluridine | 0.05% | 0.11% | 0.06% | 0.20% |
| 2′,5′- | 0.07% | 0.13% | 0.07% | 0.22% |
| diacetyluridine + | | | | |
| 3′,5′-diacetyluridine | | | | |
| Individual Unknown | ND | ND | ND | ND |
| Impurity | | | | |
| Total Impurities | 0.15% | 0.28% | 0.16% | 0.50% |
| Crystallinity | No | No | No | No |
| | Crystallinity | Crystallinity | Crystallinity | Crystallinity |
| | Detected | Detected | Detected | Detected |
| Moisture by KF | 0.88% | 0.82% | 0.80% | |

### Example 4. Treatment of Primary Mitochondrial Disease

A 10 year old male patient is diagnosed with primary mitochondrial disease by identification of pathogenic mutations known to impair mitochondrial oxidative phosphorylation (by whole exome sequencing and comparison with a database of known pathogenic genetic variants) combined with symptoms consistent with PMD, in this case including recurrent epileptic seizures, developmental delays, proximal renal tubular acidosis requiring bicarbonate supplementation, and exercise intolerance with early onset of fatigue during exertion.

Treatment with a composition of this disclosure is initiated at a dose of 60 mg/kg bodyweight of uridine triacetate, administered twice per day, before morning and evening meals. Within about one week, seizure frequency declines by more than 50%, and daily bicarbonate requirements to compensate for excessive urinary excretion due to proximal tubular acidosis is reduced from 200 milliEquivalents per day to less than 25. With the therapeutic goal of maintaining plasma bicarbonate above 20 mEq/liter. After several additional weeks, endurance during exercise improves, as measured by timed walking distance and standard clinical measures of subjective fatigue.

The dose of uridine triacetate is increased to 100 mg/kg/dose of uridine triacetate, given twice daily, and seizure frequency decreases further.

What is claimed is:

1. A composition comprising amorphous uridine triacetate dispersed in one or more excipients, wherein
the amount of the amorphous uridine triacetate is from about fifty to about sixty percent by weight of the composition;
one of the one or more excipients is Hypromellose Acetate Succinate-MG; and
the amount of the Hypromellose Acetate Succinate-MG is from about thirty-seven to about forty percent by weight of the composition.

2. The composition of claim 1, wherein one of the one or more excipients is Copovidone.

3. The composition of claim 2, wherein the amount of Copovidone is about twelve percent by weight of the composition

4. The composition of claim 1, wherein the amount of amorphous uridine triacetate is about sixty percent by weight of the composition and the amount of the Hypromellose Acetate Succinate-MG is about forty percent by weight of the composition.

5. The composition of claim 1, wherein the amount of amorphous uridine triacetate is about fifty percent by weight of the composition, and the one or more excipients comprise Hypromellose Acetate Succinate-MG in an amount of about 37.5 percent by weight of the composition and Copovidone in an amount of about 12.5 percent by weight of the composition.

6. The composition of any one of claims 1 to 5, produced by hot melt extrusion.

7. The composition of claim 6, wherein the composition comprises particles having a D50 of about 200 microns.

8. The composition of any one of claims 1 to 5, wherein the composition does not comprise a plasticizer other than the amorphous uridine triacetate.

9. A method of delivering exogenous uridine to a mammalian subject in need thereof, comprising administering to the mammalian subject an effective amount of the composition of any one of claims 1 to 5.

10. The method of claim 9, wherein the mammalian subject has a uridine deficiency condition or an energy failure disorder; or is receiving fluoropyrimidine chemotherapy and the exogenous uridine modulates toxicity or efficacy, or both toxicity and efficacy of the fluoropyrimidine chemotherapy.

11. A method for treating a mammalian subject having an energy failure disorder, comprising administering to the mammalian subject an amount of the composition of any one of claims 1 to 5 effective to treat the mammalian subject.

12. The method of claim 11, wherein the energy failure disorder is selected from the group consisting of: a primary mitochondrial disease; a chronic neurodegenerative disease featuring pathogenic deficits in mitochondrial function; a neuromuscular dysfunction or wasting featuring pathogenic mitochondrial dysfunction; an acute brain injury; a cardiac condition selected from the group consisting of myocardial ischemia and ventricular heart failure.

13. The method of claim 12, wherein the chronic neurodegenerative disease featuring pathogenic deficits in mitochondrial function is selected from the group consisting of Huntington's Disease, Alzheimer's Dementia, Parkinson's Disease, Amyotrophic Lateral Sclerosis, and Down Syndrome Dementia.

14. The method of claim 12, wherein the neuromuscular dysfunction or wasting featuring pathogenic mitochondrial dysfunction is selected from the group consisting of sarcopenia, cachexia, muscle disuse atrophy, and circulatory insufficiency.

15. The method of claim 12, wherein the acute brain injury is selected from the group consisting of traumatic brain injury, stroke, acute brain injury resulting from birth asphyxia, acute brain injury resulting from cardiac arrest, acute brain injury resulting from drowning, and acute brain injury resulting from carbon monoxide poisoning.

16. The method of claim 11, wherein the mammalian subject is a human subject and the composition is administered in one or more doses, each dose comprising from 30 to 120 milligrams of uridine triacetate per kilogram body weight of the human subject.

17. The method of claim 16, wherein the dose comprises from 60 to 100 milligrams of uridine triacetate per kilogram body weight of the human subject.

18. The method of claim 16 or 17, wherein the composition is administered orally.

19. The method of any one of claims 16 to 18, wherein from one to four doses of the composition per day are administered to the subject.

20. The method of claim 19, wherein two doses of the composition per day are administered to the subject, wherein the second and each subsequent dose in a course of treatment is administered from 8 to 12 hours after the immediately preceding administration.
